(19) United States
(12) Patent Application Publication
(10) Pub. No.: US 2021/0038909 A1
(43) Pub. Date: Feb. 11, 2021

(54) OPTICAL MEDICAL TREATMENT DEVICE

(71) Applicant: OLYMPUS CORPORATION, Tokyo (JP)

(72) Inventor: Masahiro YOSHINO, Tokyo (JP)

(73) Assignee: OLYMPUS CORPORATION, Tokyo (JP)

(21) Appl. No.: 17/078,762

(22) Filed: Oct. 23, 2020

Related U.S. Application Data
(63) Continuation of application No. PCT/JP2019/006566, filed on Feb. 21, 2019.

(30) Foreign Application Priority Data
Apr. 25, 2018 (JP) 2018-084407

(51) Int. Cl. A61N 5/06; A61B 18/24

(52) U.S. Cl. CPC A61N 5/062; A61N 2005/067; A61B 18/24

(57) ABSTRACT

An optical medical treatment device is configured as follows: a pair of electric contacts are disposed at a predetermined surface part of a catheter through which a distal end of an optical fiber configured to emit laser light is guided to an affected part inside the body of a subject, and a laser drive circuit detects whether the predetermined surface part of the catheter is in contact with inside of the body of the subject based on the state of conduction between the electric contacts, and permits emission of laser light from a laser element when at least the predetermined surface part of the catheter is in contact with inside of the body of the subject.

### CROSS REFERENCE TO RELATED APPLICATION

This application is a continuation application of PCT/JP2019/006566 filed on Feb. 21, 2019 and claims benefit of Japanese Application No. 2018-084407 filed in Japan on Apr. 25, 2018, the entire contents of which are incorporated herein by this reference.

### BACKGROUND OF THE INVENTION

### 1. Field of the Invention

The present invention relates to an optical medical treatment device configured to perform medical treatment in a body cavity through irradiation with light energy such as laser light.

### 2. Description of the Related Art

Recently, for example, photodynamic therapy (PDT) and photoimmuno therapy (PIT) have been researched in medical fields as medical treatment methods for destroying cancer cells by using high-power light energy such as laser light.

For example, as disclosed in Japanese Patent No. 6127045, the PIT includes a step of causing a therapeutically effective amount of antibody molecules to contact a cell having cell surface proteins, a step of specifically bonding an antibody to a cell surface protein such as a tumor specific antigen on the surface of a tumor cell, and a step of breaking a cell membrane by irradiating the cell with laser light having a wavelength of 660 to 740 nm and a radiation dose of at least 1 Jcm².

A laser medical treatment device used in the PIT, the PDT, and the like needs to irradiate an affected part with high-power laser light and thus needs to ensure sufficient safety by, for example, satisfying predetermined safety standards. To ensure safety, such a laser medical treatment device needs a facility environment that, for example, shields laser light to outside and prohibits laser light emission when a door is opened.

### SUMMARY OF THE INVENTION

An optical medical treatment device according to an aspect of the present invention includes: a light irradiation body configured to emit light energy to an affected part inside a body of a subject; an insertion member configured to guide the light irradiation body to the affected part; a balloon that is provided to the insertion member and into which fluid can be injected; a fluid supply device configured to supply the fluid into the balloon; a sensor system configured to detect, through a sensor provided to the insertion member, contact of a predetermined surface part of the balloon with inside of the body of the subject; and a control device configured to generate a permission signal that permits emission of the light energy when one of conditions is satisfied, the one of the conditions being detection of supply of the fluid to the balloon by the fluid supply device.

### BRIEF DESCRIPTION OF THE DRAWINGS

FIG. 1 is a block diagram illustrating a schematic configuration of a laser medical treatment device according to a first embodiment of the present invention;

FIG. 2 is a flowchart illustrating a laser emission control routine according to the first embodiment;

FIG. 3 is a main-part cross-sectional view illustrating a distal end side of a catheter before balloon expansion according to the first embodiment;

FIG. 4 is a main-part cross-sectional view illustrating the distal end side of the catheter at balloon expansion according to the first embodiment;

FIG. 5 is a main-part cross-sectional view illustrating the distal end side of the catheter at laser emission according to the first embodiment;

FIG. 6 is a block diagram illustrating a schematic configuration of the laser medical treatment device according to a first modification;

FIG. 7 is a main-part cross-sectional view illustrating the distal end side of the catheter before balloon expansion according to the first modification;

FIG. 8 is a main-part cross-sectional view illustrating the distal end side of the catheter at balloon expansion according to the first modification;

FIG. 9 is a block diagram illustrating a schematic configuration of the laser medical treatment device according to a second modification;

FIG. 10 is a schematic diagram of an image picked up from inside of a balloon according to the second modification;

FIG. 11 is a block diagram illustrating a schematic configuration of the laser medical treatment device according to a third modification;

FIG. 12 is a block diagram illustrating a schematic configuration of the laser medical treatment device according to a fourth modification;

FIG. 13 is a main-part cross-sectional view illustrating the distal end side of the catheter according to the fourth modification;

FIG. 14 is a perspective view illustrating a schematic configuration of a laser medical treatment device according to a second embodiment of the present invention;

FIG. 15 is a block diagram illustrating a schematic configuration of the laser medical treatment device according to the second embodiment;

FIG. 16 is a block diagram illustrating a schematic configuration of a laser medical treatment device according to a disclosed example; and

FIG. 17 is a schematic diagram of an endoscope image according to the disclosed example.

### DETAILED DESCRIPTION OF THE PREFERRED EMBODIMENTS

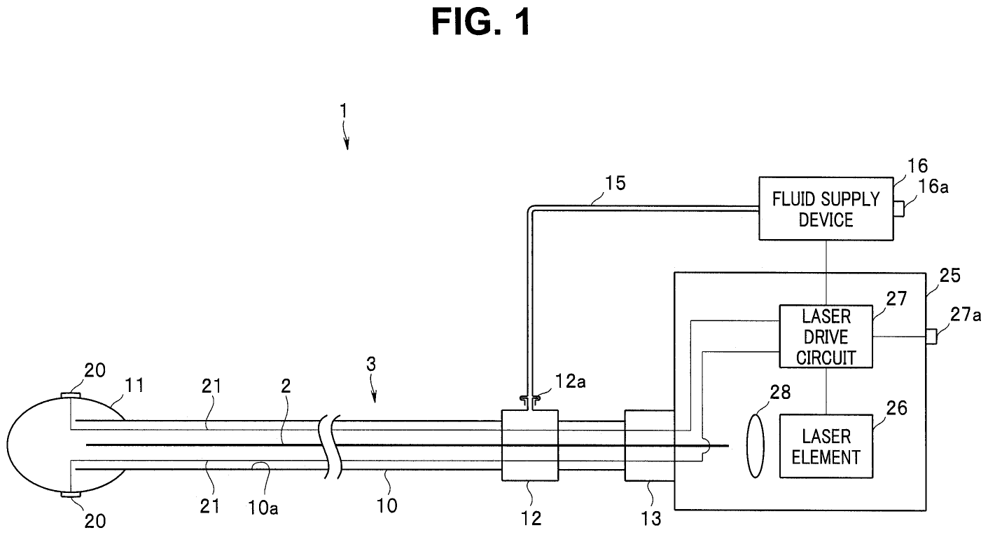

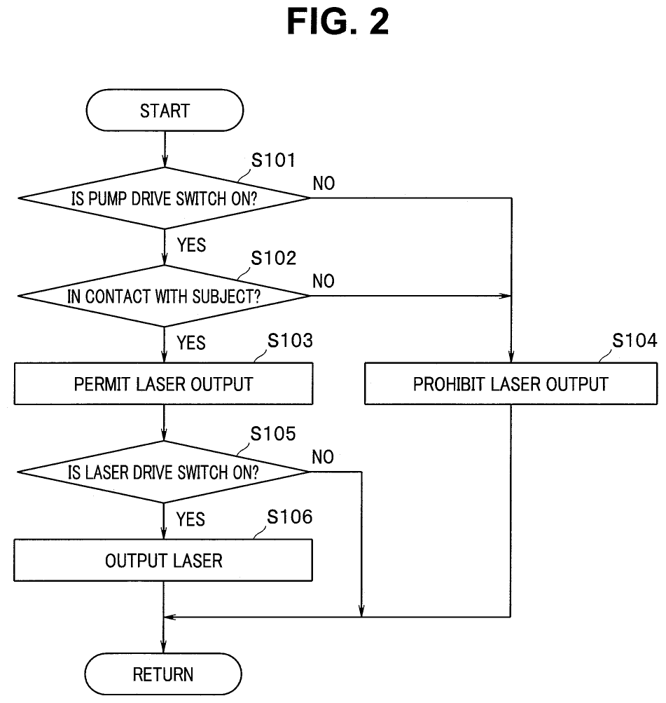

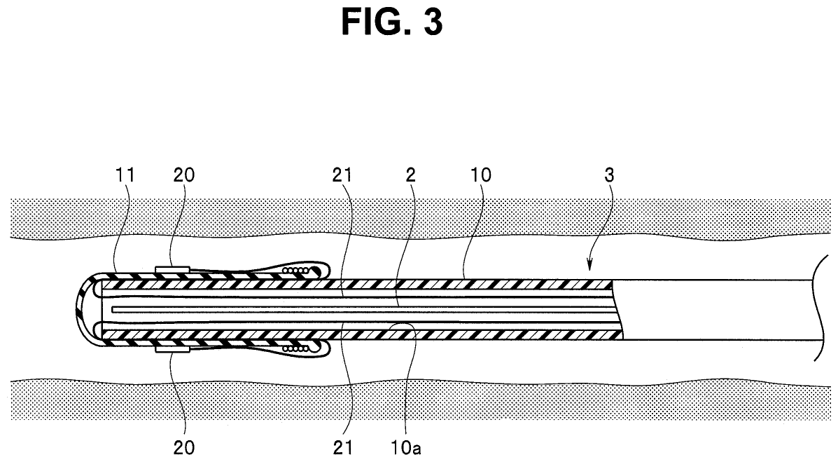

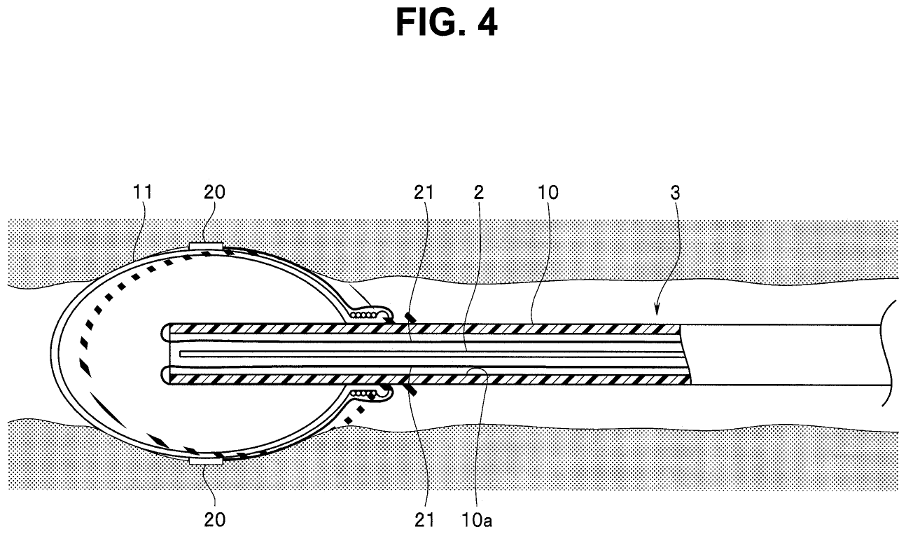

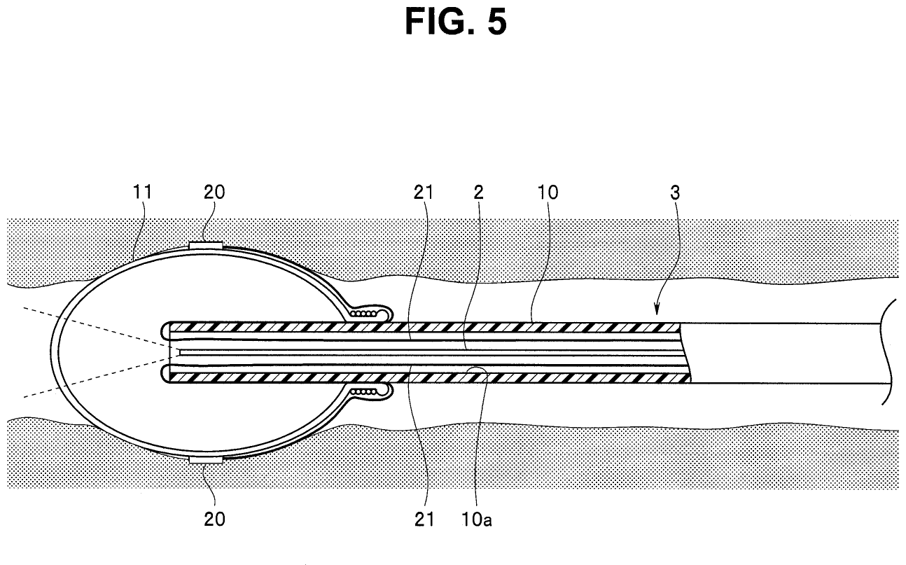

Embodiments of the present invention will be described below with reference to the accompanying drawings. FIGS. 1 to 5 relate to a first embodiment of the present invention: FIG. 1 is a block diagram illustrating a schematic configuration of a laser medical treatment device, FIG. 2 is a flowchart illustrating a laser emission control routine, FIG. 3 is a main-part cross-sectional view illustrating a distal end side of a catheter before balloon expansion, FIG. 4 is a main-part cross-sectional view illustrating the distal end side of the catheter at balloon expansion, and FIG. 5 is a main-part cross-sectional view illustrating the distal end side of the catheter at laser emission.

A laser medical treatment device 1 as an optical medical treatment device, which is illustrated in FIG. 1, irradiates an affected part in a subject with high-power laser light (near-infrared laser light) that is suitable, for example, for medical treatment such as photoimmuno therapy (PIT).

The laser medical treatment device 1 includes an optical fiber 2 as a light irradiation body configured to emit laser light to the subject, and a catheter 3 as an insertion member configured to guide the optical fiber 2 to an affected part in the body of the subject.

The catheter 3 of the present embodiment is, for example, a disposable catheter for urinary organ. The catheter 3 includes a flexible elongated catheter body 10 that can be inserted into the body of the subject.

The catheter body 10 is configured by, for example, a single lumen tube including a single pipe line 10a into which the optical fiber 2 can be inserted.

A balloon 11 is provided at a distal end of the catheter body 10. The balloon 11 has a bag shape and is disposed to cover the distal end of the catheter body 10. An open end part of the balloon 11 is fixed to an outer peripheral surface of the catheter body 10 in a liquid-tight manner by winding bonding or the like. A distal end opening part of the pipe line 10a formed in the catheter body 10 is blocked in a liquid-tight manner by the balloon 11 thus fixed.

The balloon 11 is formed of an elastic body such as rubber, which has a transparent property to laser light emitted from the optical fiber 2.

A bifurcation connector 12 for bifurcating halfway through the pipe line 10a is provided at a proximal end side of the catheter body 10.

A bifurcation port 12a provided at the bifurcation connector 12 is connected with a downstream end of a fluid pipe line 15. A fluid supply device 16 of a pump type, for example, is connected with an upstream end of the fluid pipe line 15.

The fluid supply device 16 is provided with a pump drive switch 16a, and when the pump drive switch 16a is turned on, the fluid supply device 16 can compress fluid such as normal saline solution and supply the fluid into the pipe line 10a of the catheter body 10. By supplying the fluid to the pipeline 10a in this manner, the fluid supply device 16 can expand the balloon 11.

As illustrated in, for example, FIGS. 1 and 4, the balloon 11 is provided with a pair of electric contacts 20 at positions separated from each other at a surface part (maximum outer diameter portion) that has a maximum outer diameter at expansion. Each electric contact 20 is connected with a distal end side of a signal line 21 inserted in the catheter body 10.

A light source connector 13 for detachably connecting the catheter 3 to a light source device 25 is provided at a proximal end of the catheter body 10.

A proximal end side of the optical fiber 2 is penetrated through and fixed to the light source connector 13. Accordingly, when connected with the light source device 25, the light source connector 13 can position a proximal end of the optical fiber 2 at a predetermined position inside the light source device 25. The light source connector 13 is electrically connected with the other end of each signal line 21 inserted in the pipe line 10a of the catheter body 10.

The light source device 25 includes a laser element 26 and a laser drive circuit 27.

The laser element 26 is configured of, for example, a laser diode capable of emitting near-infrared laser light. The laser element 26 is disposed at a position facing, through a lens 28, the proximal end of the optical fiber 2 positioned inside the light source device 25. Accordingly, the laser element 26 can emit laser light into the optical fiber 2.

When the light source connector 13 is connected with the light source device 25, the laser drive circuit 27 is connected with the pair of signal lines 21 inserted in the catheter body 10. Then, the laser drive circuit 27 monitors electrical connection between the electric contacts 20 connected with the of pair signal lines 21, thereby detecting whether the circumference of the balloon 11 provided to the catheter 3 is substantially entirely in contact with inside of the body of the subject.

Specifically, as illustrated in, for example, FIG. 3, the pair of signal lines 21 are electrically cut off, even when the catheter 3 is inserted in the body of the subject, if the pair of electric contacts 20 are not simultaneously in contact with a body wall of the subject. Thus, even if the laser drive circuit 27 applies an examination signal to one of the signal lines 21, the examination signal cannot be detected at the other signal line 21. However, when the circumference of the balloon 11 is substantially entirely in contact with the body wall of the subject by expansion of the balloon 11 as illustrated in, for example, FIG. 4, the pair of electric contacts 20 are simultaneously in contact with the body wall of the subject and electrically connected with each other through mucus or the like of the subject. Accordingly, the pair of signal lines 21 form a closed circuit, and the laser drive circuit 27 can detect, as a detection signal indicating contact of the catheter 3 with inside of the body of the subject, an examination signal applied to one of the signal lines 21 at the other signal line 21.

In this manner, in the present embodiment, the pair of electric contacts 20 achieve functions of sensors (contact sensing sensors) for detecting, at a plurality of places (two places or more), contact of an outer periphery of the balloon 11 with inside of the body of the subject. In addition, the laser drive circuit 27 achieves, together with the pair of electric contacts 20 and the signal lines 21, functions of a sensor system configured to generate a detection signal when a predetermined surface part of the catheter 3 contacts inside of the body of the subject.

The sensor system in this case may have a configuration in which distortion sensors or the like as sensors are disposed at a plurality of places on the outer periphery of the balloon 11 in place of the electric contacts 20 so that the laser drive circuit 27 individually detects signals outputted from the distortion sensors or the like upon contact with inside of the body of the subject.

The laser drive circuit 27 receives an on-off signal for the pump drive switch 16a from the fluid supply device 16, and an on-off signal from a laser drive switch 27a provided to the light source device 25.

Then, for example, when a condition is satisfied that the pump drive switch 16a is on and contact between inside of the body of the subject and the catheter 3 (specifically, a surface part of the balloon 11 as the predetermined surface part of the catheter 3) is detected, the laser drive circuit 27 performs determination to permit laser light emission. Only when the laser drive switch 27a is turned on during the permission determination, the laser drive circuit 27 drives the laser element 26 to emit laser light (refer to FIG. 5).

In other words, since a distal end of the optical fiber 2, from which laser light is emitted, is highly likely to be out of the body of the subject in a case in which the pump drive switch 16a is off or contact of the catheter 3 with inside of the body of the subject is not detected, the laser drive circuit 27 prohibits laser light emission even when the laser drive switch 27a is turned on in this case.

In this manner, the laser drive circuit 27 has functions of a control device configured to control laser emission and stop. In the above-described permission determination of laser light emission, determination based on the pump drive switch 16a may be omitted as appropriate.

The laser emission control by the laser drive circuit 27 will be described below in accordance with a flowchart of the laser emission control routine illustrated in FIG. 2.

The routine is repeatedly executed at each set time. When the routine is started, the laser drive circuit 27 first determines whether the pump drive switch 16a is on at step S101.

When having determined that the pump drive switch 16a is on at step S101, the laser drive circuit 27 proceeds to step S102. When having determined that the pump drive switch 16a is off, the laser drive circuit 27 proceeds to step S104.

When having proceeded to step S102 from step S101, the laser drive circuit 27 determines whether the balloon 11 is in contact with a predetermined part in the body of the subject based on the state of energization between the signal lines 21 (in other words, whether an examination signal is detected).

When having determined that the balloon 11 is in contact with the predetermined part in the body of the subject at step S102, the laser drive circuit 27 proceeds to step S103. When having determined that the balloon 11 is not in contact with the predetermined part in the body of the subject, the laser drive circuit 27 proceeds to step S104.

When having proceeded to step S104 from step S101 or S102, the laser drive circuit 27 performs determination to prohibit laser light outputting from the laser element 26, and then leaves the routine.

When proceeding to step S105 from step S103, the laser drive circuit 27 performs determination to permit laser light outputting from the laser element 26, and then proceeds to step S105.

Then, at step S105, the laser drive circuit 27 determines whether the laser drive switch 27a is on. When having determined that the laser drive switch 27a is off, the laser drive circuit 27 directly leaves the routine without outputting laser light.

When having determined that the laser drive switch 27a is on at step S105, the laser drive circuit 27 proceeds to step S106 to perform drive control for outputting laser light from the laser element 26, and then leaves the routine.

According to such an embodiment, the pair of electric contacts 20 are disposed at the predetermined surface part of the catheter 3 through which the distal end of the optical fiber 2 configured to emit laser light is guided to an affected part in the body of the subject, and the laser drive circuit 27 detects whether the predetermined surface part of the catheter 3 is in contact with inside of the body of the subject based on the state of conduction between the electric contacts 20, and permits laser light emission from the laser element 26 when at least the predetermined surface part of the catheter 3 is in contact with inside of the body of the subject. In this manner, it is possible to emit high-power light energy into the body cavity while ensuring safety with a simple configuration.

Accordingly, it is possible to accurately determine whether the catheter 3 for guiding the distal end of the optical fiber 2 into the body of the subject is inserted in the body of the subject by detecting the state of contact of the predetermined surface part of the catheter 3 with inside of the body of the subject. Then, when the catheter 3 is in contact with inside of the body of the subject (in other words, when the catheter 3 is inserted in the body of the subject), it is determined that the distal end of the optical fiber 2 is inserted in the body of the subject and laser light emission is permitted, thereby accurately preventing laser light emission out of the body of the subject. Thus, when high-power laser light is used, as well, it is possible to ensure safety without providing a dedicated medical treatment room or the like.

In this case, since the balloon 11 is provided at a distal end of the catheter 3 and the pair of electric contacts 20 are provided at a maximum outer diameter portion that has a maximum outer diameter when the balloon 11 is inflated, a user such as an operator can explicitly perform an operation for inflating the balloon 11 and making the balloon 11 contact with inside of the body of the subject, and then determine whether a surface part of the balloon 11 is in contact with inside of the body of the subject. Thus, it is possible to more accurately perform the determination whether the catheter 3 is inserted in the body of the subject based on the contact with inside of the body of the subject.

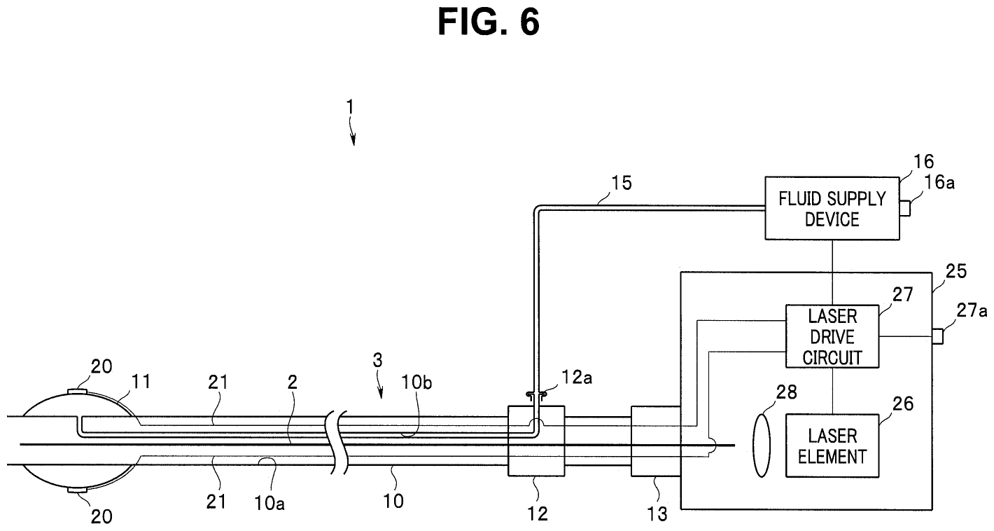

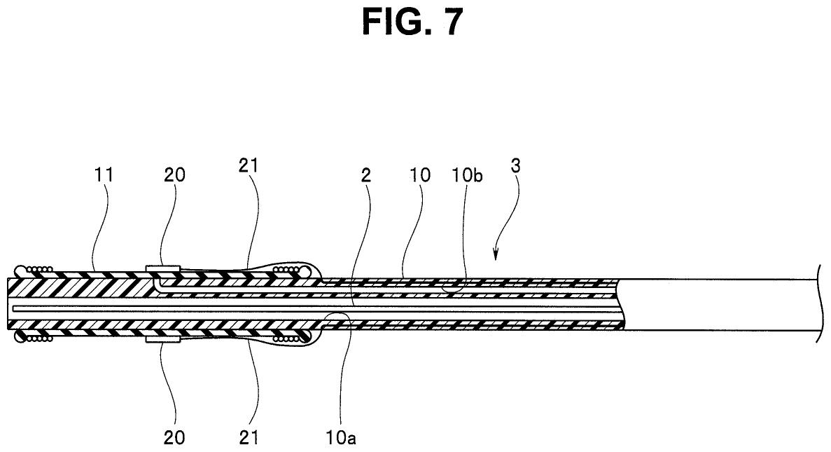

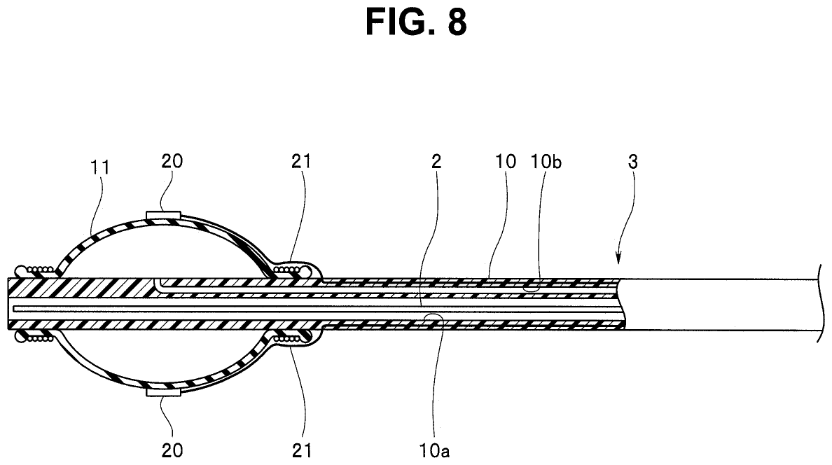

A balloon 11 formed in a cylindrical shape, for example, as illustrated in FIGS. 6 to 8 may be employed as a balloon provided at the distal end of the catheter 3 in place of the balloon 11 formed in a bag shape.

In the catheter 3 provided with such a balloon 11, the catheter body 10 is configured by a multi lumen tube including a pipe line 10b through which fluid is circulated in addition to the pipe line 10a in which the optical fiber 2 is inserted.

Both end parts of the balloon 11 are fixed to the outer peripheral surface of the catheter body 10 in a liquid-tight manner by winding bonding or the like. A closed space formed by such fixation of the balloon 11 communicates with a distal end of the pipe line 10b.

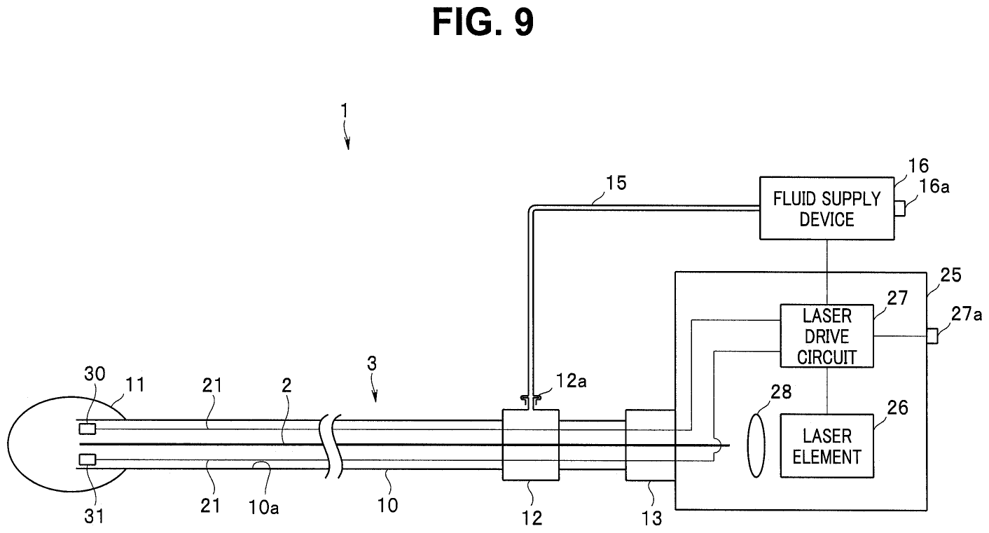

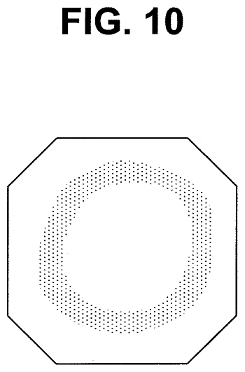

As illustrated in, for example, FIGS. 9 and 10, the sensor system may employ a configuration including, as sensors, an image sensor 30 such as a CCD or a CMOS, and a light source 31 such as an LED in place of a configuration including electrodes and the like as described above.

In the catheter 3 thus configured, the light source 31 is disposed inside the balloon 11 closed in a bag shape at a position where the light source 31 can emit illumination light through an illumination optical system (not illustrated).

The image sensor 30 is disposed at a position where the image sensor 30 can pick up an image of inside of the balloon 11 closed in a bag shape through an image pickup optical system (not illustrated).

The laser drive circuit 27 performs image recognition on the image picked up by the image sensor 30 to determine whether the predetermined surface part of the catheter 3 (in other words, the balloon 11) is in contact with inside of the body of the subject.

Specifically, when the outer periphery of the balloon 11 being inflated is in contact with the body wall of the subject or the like, this contact part is picked up as a region in a ring shape more strongly affected by color and the like of the body wall than the other part as illustrated in, for example, FIG. 10. Thus, it is possible to determine whether the predetermined surface part of the balloon 11 is in contact with inside of the body of the subject by determining the existence of such a region through pattern recognition or the like by the laser drive circuit 27.

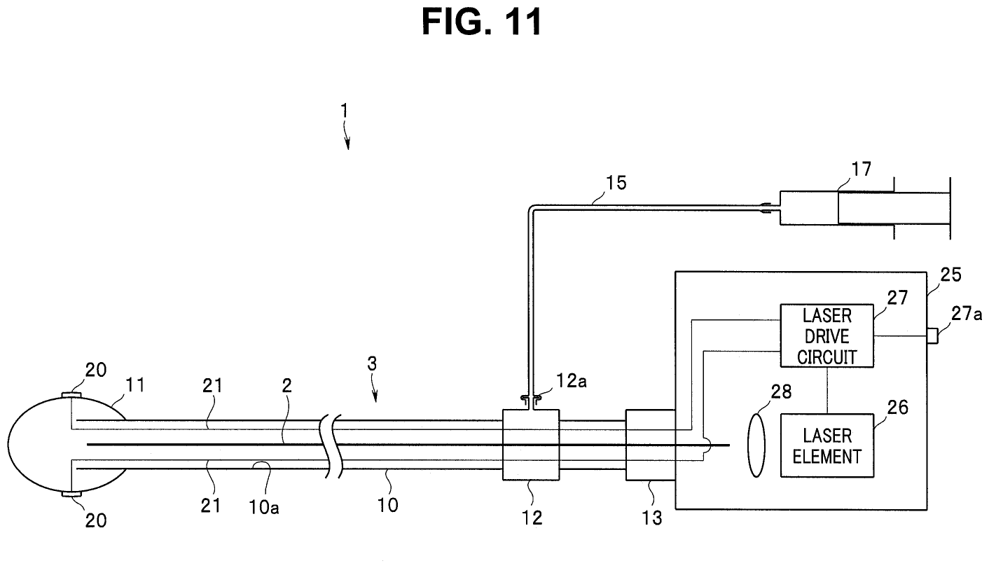

In the above-described embodiment and modifications, as illustrated in, for example, FIG. 11, the fluid pipe line 15 may be connected with a syringe 17 in place of the fluid supply device 16 to perform fluid supply to the balloon 11 by using the syringe 17.

In a modification having such a configuration, fluid supply to the balloon 11 is manually performed by the operator or the like. Thus, laser emission control executed at the laser drive circuit 27 is the laser emission control described above with reference to FIG. 2 from which the determination at step S101 is omitted. Specifically, when the routine is started, the laser drive circuit 27 performs the above-described determination at step S102 as the first processing.

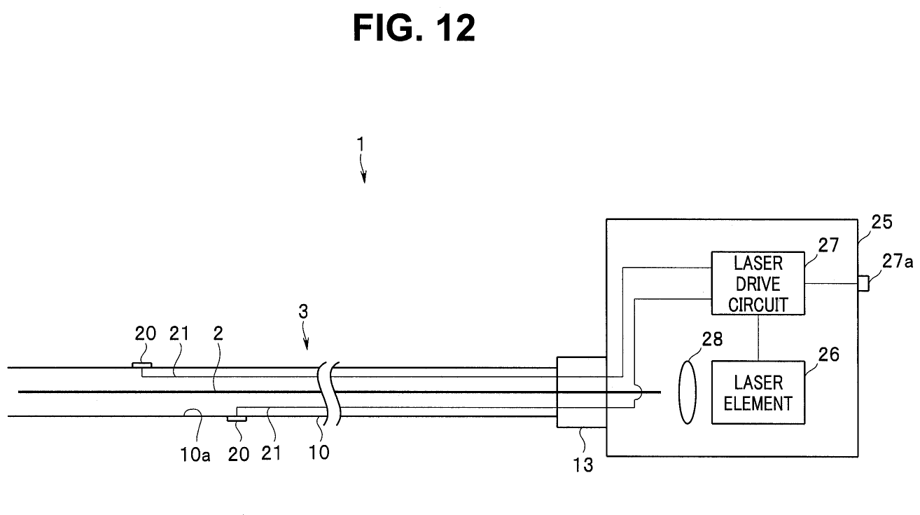

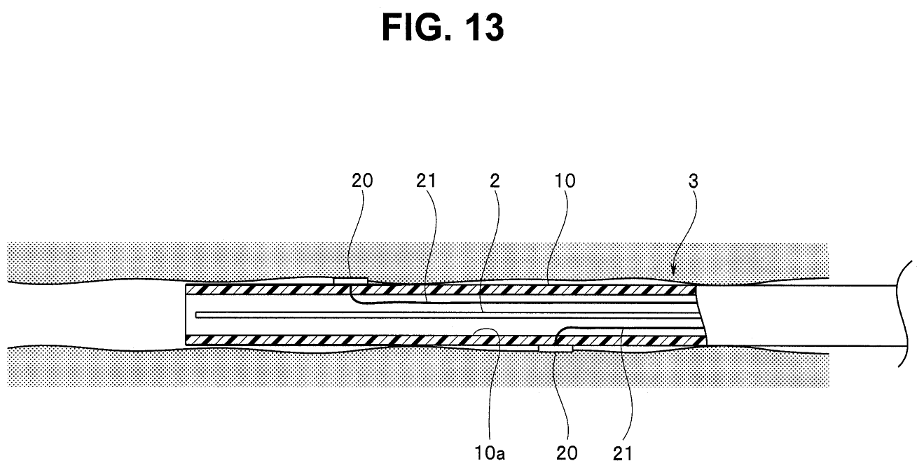

As illustrated in, for example, FIGS. 12 and 13, when the catheter 3 is inserted into a lumen having a diameter smaller than that of a ureter or the like, the balloon may be removed and the pair of electric contacts 20 may be directly provided as sensors on an outer surface of the catheter body 10.

In this case, the electric contacts 20 are desirably separated from each other by a predetermined distance in a longitudinal direction of the catheter body 10 to prevent a hand of the operator or the like from simultaneously contacting the pair of electric contacts 20, for example, before insertion into the body of the subject.

In a modification having such a configuration, as well, since no balloon is provided, laser emission control executed at the laser drive circuit 27 is the laser emission control described above with reference to FIG. 2 from which the determination at step S101 is omitted.

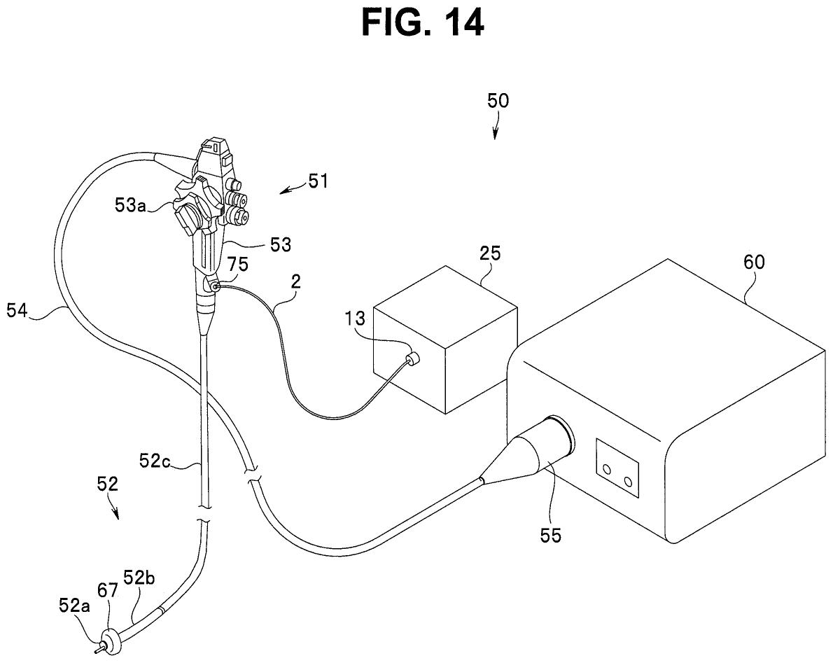

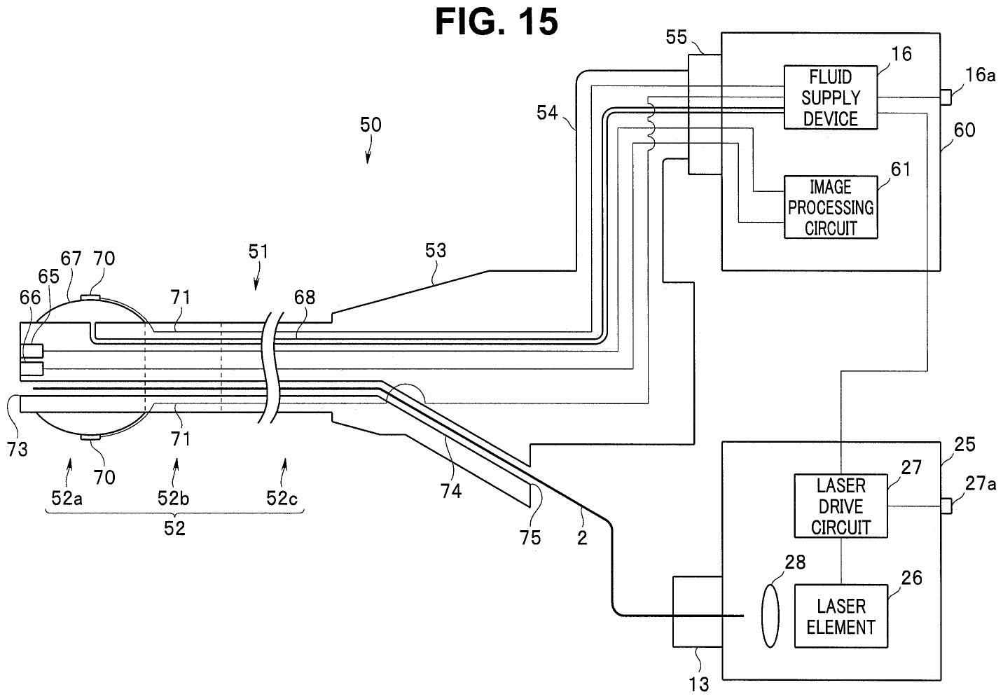

FIGS. 14 and 15 relate to a second embodiment of the present invention: FIG. 14 is a perspective view illustrating a schematic configuration of a laser medical treatment device, and FIG. 15 is a block diagram illustrating a schematic configuration of the laser medical treatment device. The present embodiment is different from that of the first embodiment described above mainly in that an endoscope is used as an insertion member in place of the catheter. Thus, a component same as that of the first embodiment described above is denoted by the same reference sign as appropriate, and description thereof is omitted.

A laser medical treatment device 50 illustrated in FIGS. 14 and 15 includes the optical fiber 2 as a light irradiation body configured to emit laser light to a subject, and an endoscope 51 as an insertion member configured to guide the optical fiber 2 into the body of the subject.

The light source connector 13 for detachably connecting the optical fiber 2 to the light source device 25 is provided at the proximal end side of the optical fiber 2. Accordingly, when connected with the light source device 25, the light source connector 13 can position the proximal end of the optical fiber 2 at a predetermined position inside the light source device 25.

The endoscope 51 includes an elongated insertion portion 52 that is inserted into the subject, an operation portion 53 provided at a rear end of the insertion portion 52, serving as a grasping portion, and configured to perform various operations, and a universal code 54 extending from the operation portion 53. The endoscope 51 is detachably attached to a processor 60 through an endoscope connector 55 provided at an end part of the universal code 54.

The insertion portion 52 has a configuration in which a rigid distal end portion 52a provided on a distal end side, a bending portion 52b that is bendable and provided continuously with a rear end of the distal end portion 52a, and a flexible pipe portion 52c provided from a rear end of the bending portion 52b to a front end of the operation portion 53 are continuously provided.

The distal end portion 52a includes a light source 65 configured to emit illumination light to an observation site through an illumination optical system (not illustrated), and an image sensor 66 as a sensor configured to pick up an image of the observation site through an image pickup optical system (not illustrated).

A cylindrical balloon 67 is provided at an outer periphery of the distal end portion 52a. A distal end side and a proximal end side of the balloon 67 are fixed to the outer periphery of the distal end portion 52a in a liquid-tight manner by winding bonding or the like. A closed space formed by such fixation of the balloon 67 communicates with a pipe line 68.

The balloon 67 is provided with a pair of electric contacts 70 at a maximum outer diameter portion that has a maximum outer diameter when the balloon 67 is inflated, and each electric contact 70 is connected with a signal line 71 inserted in the insertion portion 52.

A distal end opening part 73 is provided at the distal end portion 52a. The distal end opening part 73 communicates with, through a channel tube 74, a treatment instrument insertion opening 75 provided at the operation portion 53. The optical fiber 2 can be inserted through the treatment instrument insertion opening 75, and accordingly, the distal end of the optical fiber 2 can be guided into the body of the subject through the insertion portion 52 of the endoscope 51.

A plurality of bent pieces are disposed at the bending portion 52b and can be driven by a bending wire coupled with a bending operation knob 53a provided at the operation portion 53, thereby bending the bending portion 52b in a desired direction.

The processor 60 includes an image processing circuit 61 and the fluid supply device 16.

The image processing circuit 61 can control drive of the light source 65 and the image sensor 66 and display an image picked up by the image sensor 66 on a monitor (not illustrated).

When the pump drive switch 16a is turned on, the fluid supply device 16 can compress fluid such as normal saline solution and supply the fluid into the pipe line 68 of the endoscope 51. By supplying the fluid to the pipe line 68 in this manner, the fluid supply device 16 can expand the balloon 67.

In addition, in place of the laser drive circuit 27, the fluid supply device 16 of the present embodiment achieves, together with the pair of electric contacts 70 and the signal lines 71, functions of a sensor system configured to generate a detection signal when a predetermined surface part of the endoscope 51 contacts inside of the body of the subject. Detection of the contact with inside of the body of the subject is same as the processing performed by the laser drive circuit 27 in the first embodiment described above, and thus description thereof is omitted.

According to the second embodiment having such a configuration, it is possible to achieve effects substantially same as those of the first embodiment described above.

The present invention is not limited to the embodiments and modifications described above but may be provided with deformations and changes in various manners, which are included in the technical scope of the present invention. For example, components of the above-described embodiments may be combined as appropriate.

For example, in place of an optical fiber, a light-emitting element configured to emit high-power near-infrared light may be disposed at a distal end of a catheter or the like to irradiate an affected part with light energy other than laser light as medical-treatment light energy.

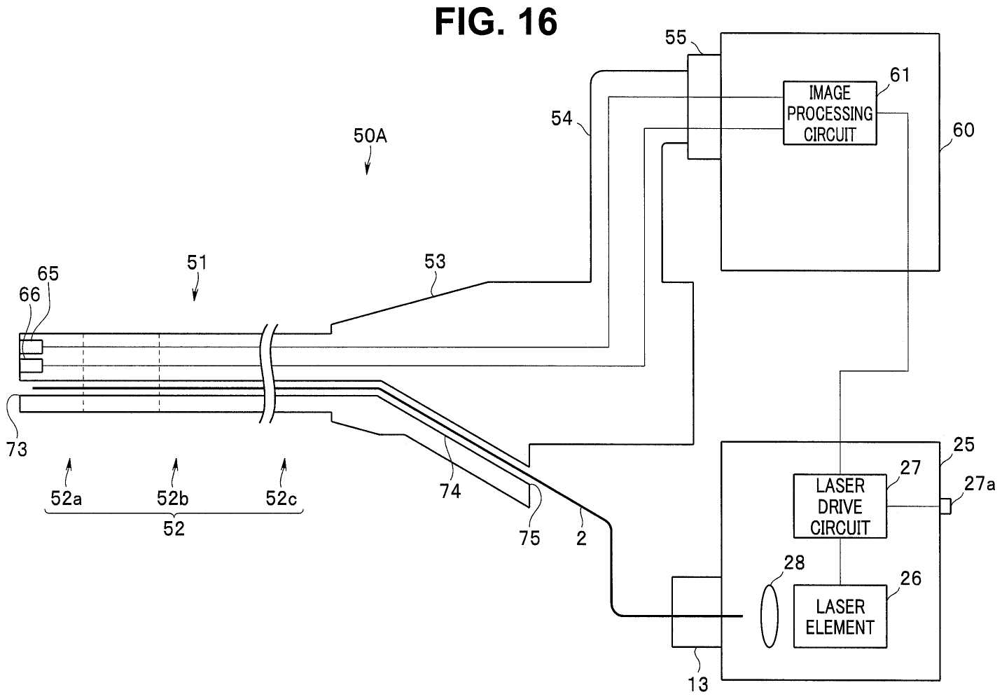

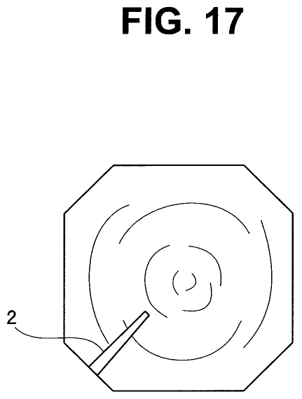

FIGS. 16 and 17 relate to a disclosed example of the present invention: FIG. 16 is a block diagram illustrating a schematic configuration of a laser medical treatment device, and FIG. 17 is a schematic diagram of an endoscope image.

A laser medical treatment device 50A of the present disclosed example illustrated in FIG. 16 has the above-described configuration of the second embodiment from which the fluid supply device 16, the balloon 67, the electric contacts 70, the signal line 71, and the like are removed.

In the disclosed example, publicly known pattern matching or the like is performed based on image information (refer to FIG. 17) picked up by the image sensor 66 to determine whether the insertion portion 52 of the endoscope 51 is inserted in a subject, and laser light emission from the optical fiber 2 inserted in the channel tube 74 (laser light emission from the laser element 26) is permitted when it is determined that the insertion portion 52 is inserted in the subject.

What is claimed is:

1. An optical medical treatment device comprising:
a light irradiation body configured to emit light energy to an affected part inside a body of a subject;
an insertion member configured to guide the light irradiation body to the affected part;
a balloon that is provided to the insertion member and into which fluid can be injected;
a fluid supply device configured to supply the fluid into the balloon;
a sensor system configured to detect, through a sensor provided to the insertion member, contact of a predetermined surface part of the balloon with inside of the body of the subject; and
a control device configured to generate a permission signal that permits emission of the light energy when one of conditions is satisfied, the one of the conditions being detection of supply of the fluid to the balloon by the fluid supply device.

2. The optical medical treatment device according to claim 1, wherein the conditions further include detection of contact of the predetermined surface part of the balloon with inside of the body of the subject by the sensor.

3. The optical medical treatment device according to claim 1, wherein the sensor system includes, as the sensor, a contact sensing sensor disposed on the predetermined surface part of the balloon.

4. The optical medical treatment device according to claim 3, wherein the contact sensing sensor is disposed at each of a plurality of places.

5. The optical medical treatment device according to claim 4, wherein the contact sensing sensor at each of the plurality of places is disposed at a maximum outer diameter portion that has a maximum outer diameter when the balloon is inflated.

6. The optical medical treatment device according to claim 1, wherein the sensor system includes, as the sensor, an image sensor disposed in the insertion member.

7. The optical medical treatment device according to claim 6, wherein the image sensor is disposed to pick up an image of a part at which inside of the body contacts the balloon being inflated.
